(19) United States
(12) Patent Application Publication
(10) Pub. No.: US 2024/0074807 A1
(43) Pub. Date: Mar. 7, 2024

(54) MICRONEEDLE THERAPY CONTROL METHOD AND DEVICE, AND RADIO-FREQUENCY MICRONEEDLE THERAPY INSTRUMENT

(71) Applicant: SHENZHEN PENINSULA MEDICAL GROUP, Shenzhen (CN)

(72) Inventors: Xiaobing LEI, Shenzhen (CN); Yanan LI, Shenzhen (CN); Yongsheng LIANG, Shenzhen (CN); Zhikuan XIA, Shenzhen (CN)

(73) Assignee: SHENZHEN PENINSULA MEDICAL GROUP, Shenzhen (CN)

(21) Appl. No.: 18/505,798

(22) Filed: Nov. 9, 2023

Related U.S. Application Data
(63) Continuation of application No. PCT/CN2021/128206, filed on Nov. 2, 2021.

(30) Foreign Application Priority Data
Nov. 4, 2020 (CN) 202011221474.0

(51) Int. Cl. A61B 18/12; A61B 18/14

(52) U.S. Cl. CPC A61B 18/1206; A61B 18/1477; A61B 2018/0047

(57) ABSTRACT

Disclosed are a microneedle therapy control method and a microneedle therapy control device, and a radio-frequency (RF) microneedle therapy instrument. The microneedle therapy control method comprises the following steps: acquiring a piercing depth of a microneedle electrode; determining, according to the piercing depth of the microneedle electrode, a movement interval of the microneedle electrode in a length direction; sending a first control signal to a linear driver, so as to enable the linear driver to drive the microneedle electrode to move at a preset speed along the length direction within the movement interval; and sending a second control signal to a power module, so as to enable the power module to provide RF energy to the microneedle electrode during the movement of the microneedle electrode in the movement interval.

### CROSS-REFERENCE TO RELATED APPLICATIONS

This application is a continuation of International Application No. PCT/CN2021/128206, filed on Nov. 2, 2021, which claims priority to Chinese Patent Application No. 202011221474.0, filed on Nov. 4, 2020. All of the aforementioned applications are incorporated herein by reference in their entireties.

### TECHNICAL FIELD

The present application relates to the technical field of medical instrument, in particular to a microneedle therapy control method, a device and a radio-frequency microneedle therapy instrument.

### BACKGROUND

Radio-frequency microneedle therapy is a micro-invasive radiofrequency array technology that uses tiny microneedles to precisely deliver radio-frequency (RF) energy to target tissues at different depths, which can be applied to facial rejuvenation, such as skin tightening and scar removal, and can also applied to the acne therapy and the axillary hyperhidrosis therapy.

However, the existing radio-frequency therapy instrument has poor therapy effects on target tissues with a long spreading area in depth.

### SUMMARY

The main purpose of this application is to provide a microneedle therapy control method, a device and a radio-frequency microneedle therapy instrument, aiming to solve the problem that the existing radio-frequency therapy instrument has poor therapy effects on target tissues with a long spreading area in depth.

In order to achieve the above objectives, the present application provides a microneedle therapy control method, applied to a radio-frequency microneedle therapy instrument. The radio-frequency microneedle therapy instrument includes:
at least two microneedle electrodes movable along a length direction of the microneedle electrodes;
a power supply module configured to supply radio-frequency energy for the microneedle electrode; and
a linear driver configured to drive the microneedle electrode to move.

The method includes:
obtaining a penetration depth of the microneedle electrode;
determining a movement range of the microneedle electrode in the length direction according to the penetration depth of the microneedle electrode;
sending a first control signal to the linear driver, to allow the linear driver to drive the microneedle electrode to move along the length direction at a preset speed within the movement range; and
sending a second control signal to the power supply module, to allow the power supply module to supply radio-frequency energy for the microneedle electrode during a movement of the microneedle electrode within the movement range.

In an embodiment, the sending the second control signal to the power supply module, to allow the power supply module to supply radio-frequency energy for the microneedle electrode during the movement of the microneedle electrode within the movement range includes:
sending the second control signal to the power supply module, to allow the power supply module to supply one of a first electric polarity and a second electric polarity for at least one of the at least two microneedle electrodes during the movement of the microneedle electrode within the movement range, and allow the power supply module to supply another one of the first electric polarity and the second electric polarity for remaining microneedle electrodes of the at least two microneedle electrodes.

In an embodiment, the sending the first control signal to the linear driver, to allow the linear driver to drive the microneedle electrode to move along the length direction at the preset speed within the movement range includes:
sending the first control signal to the linear driver, to allow the linear driver to drive the microneedle electrode to move in an outward direction from a human body at a preset speed within the movement range.

In an embodiment, the sending the second control signal to the power supply module, to allow the power supply module to supply radio-frequency energy for the microneedle electrode during the movement of the microneedle electrode within the movement range includes:
determining a preset operating power mode of the power supply module according to a mapping table between a part to be treated and a preset power mode of the power supply module, the preset power mode includes an increasing power mode, a constant power mode and a decreasing power mode; and
sending the second control signal to the power supply module, to allow the power supply module to operate in the preset power mode and supply radio-frequency energy for the microneedle electrode during the movement of the microneedle electrode within the movement range.

In an embodiment, before the sending the first control signal to the linear driver, to allow the linear driver to drive the microneedle electrode to move along the length direction at the preset speed within the movement range, the control method further includes:
sending a third control signal to the linear driver according to the penetration depth, to allow the linear driver to drive the microneedle electrode to penetrate into the human body until a needle tip of the microneedle electrode reaches the penetration depth; and
sending a fourth control signal to the power supply module, to allow the power supply module to supply radio-frequency energy for at least part of the at least two microneedle electrodes within a preset duration.

In an embodiment, the radio-frequency microneedle therapy instrument further includes a return electrode arranged at a body surface, the return electrode is electrically connected to the power supply module; and
the sending the fourth control signal to the power supply module, to control the power supply module to supply radio-frequency energy for at least part of the at least two microneedle electrodes within the preset duration includes:
sending the fourth control signal to the power supply module, to keep the power supply module in a unipolar mode within the preset duration to supply one of a first electric polarity and a second electric polarity for the at least two microneedle electrodes and supply another one of the first electric polarity and the second electric polarity for the return electrode.

In an embodiment, the fourth control signal is further configured to trigger the power supply module to be in a bipolar mode during a conversion duration, to supply one of the first electric polarity and the second electric polarity for at least one of a plurality of microneedle electrodes and supply another one of the first electric polarity and the second electric polarity for remaining microneedle electrodes of the plurality of the microneedle electrodes, the conversion duration is a part of the preset duration, and the unipolar mode and the bipolar mode are switched at least once within the preset duration.

In an embodiment, the determining the movement range of the microneedle electrode in the length direction according to the penetration depth of the microneedle electrode includes:
determining a minimum energy output depth of the microneedle electrode according to the part to be treated; and
determining the movement range of the microneedle electrode in the length direction according to the penetration depth and the minimum energy output depth.

In an embodiment, the obtaining the penetration depth of the microneedle electrode includes:
obtaining the penetration depth of the microneedle electrode according to a mapping table between the part to be treated and a preset penetration depth of the microneedle electrode.

In a second aspect, the present application further provides a microneedle therapy control device, including:
a depth obtaining module, configured to obtain a penetration depth of a microneedle electrode;
a range determination module, configured to determine a movement range of the microneedle electrode in a length direction according to the penetration depth of the microneedle electrode; and
a signal sending module, configured to send a first control signal to a linear driver to allow the linear driver to drive the microneedle electrode to move along a length direction at a preset speed within the movement range, and send a second control signal to a power supply module to allow the power supply module to supply radio-frequency energy for the microneedle electrode during a movement of the microneedle electrode within the movement range.

In a third aspect, the present application further provides a radio-frequency microneedle therapy instrument, including:
at least two microneedle electrodes movable along a length direction of the microneedle electrodes;
a power supply module configured to supply radio-frequency energy for the microneedle electrode;
a linear driver configured to drive the microneedle electrode to move; and
at least one processor, a memory, and a microneedle therapy control program stored in the memory and executable on the processor, when the microneedle therapy control program is executed by the processor, the microneedle therapy control method is implemented.

The present application provides a microneedle therapy control method, a linear driver is controlled to drive the microneedle electrode to move along the depth direction of the target tissue, and the microneedle electrode continuously releases radio-frequency energy during the movement process, to effectively treat all parts of the target tissue. In this way, not only the target tissue at the penetration depth can be treated, but also the target tissue at a smaller depth can be treated, thereby improving the effect of radio-frequency therapy.

### BRIEF DESCRIPTION OF THE DRAWINGS

To illustrate the technical solutions according to the embodiments of the present application or the related art more clearly, the accompanying drawings for describing the embodiments or the related art are introduced briefly in the following. Apparently, the accompanying drawings in the following description are only some embodiments in the present application. Person of ordinary skill in the art can derive other drawings from the structures in the accompanying drawings without creative efforts.

FIG. 1 is a schematic structural diagram of a radio-frequency therapy instrument in the present application.

FIG. 2 is a schematic flowchart of a microneedle therapy control method according to a first embodiment of the present application.

FIG. 3 is a schematic flowchart of the microneedle therapy control method according to a third embodiment of the present application.

FIG. 4 is a schematic view of a penetration depth of a microneedle electrode in the present application.

FIG. 5 is a structural block diagram of a microneedle therapy control device according to a first embodiment of the present application.

The realization of the objective, functional characteristics, and advantages of the present application are further described with reference to the embodiments and the accompanying drawings.

### DETAILED DESCRIPTION OF THE EMBODIMENTS

The technical solutions of the embodiments of the present application will be described in detail below with reference to the accompanying drawings. It is obvious that the embodiments described are only some rather than all of the embodiments of the present application. All other embodiments obtained by those skilled in the art according to the embodiments of the present application without creative efforts shall fall within the claimed scope of the present application.

It should be noted that all the directional indications (such as up, down, left, right, front, rear . . . ) in the embodiments of the present application are only used to explain the relative positional relationship, movement, or the like of the components in a certain posture (as shown in the drawings). If the specific posture changes, the directional indication will change accordingly.

In the present application, unless otherwise clearly specified and limited, the terms “connected”, “fixed”, etc. should be interpreted broadly. For example, “fixed” can be a fixed connection, a detachable connection, or a whole; can be a mechanical connection or an electrical connection; may be directly connected, or indirectly connected through an intermediate medium, and may be the internal communication between two elements or the interaction relationship between two elements, unless specifically defined otherwise. For those of ordinary skill in the art, the specific meaning of the above-mentioned terms in the present application can be understood according to specific circumstances.

Besides, the descriptions associated with, e.g., “first” and “second”, in the present application are merely for descriptive purposes, and cannot be understood as indicating or suggesting relative importance or impliedly indicating the number of the indicated technical feature. Therefore, the feature associated with “first” or “second” can expressly or impliedly include at least one such feature. In addition, the meaning of “and/or” appearing in the whole application includes three parallel schemes. For example, “A and/or B” includes scheme A, scheme B, or schemes that both A and B satisfy. Moreover, the technical solutions of the various embodiments can be combined with each other, but the combinations must be according to the realization of those skilled in the art. When the combination of technical solutions is contradictory or cannot be achieved, it should be considered that such a combination of technical solutions does not exist, nor does it fall within the scope of the present application.

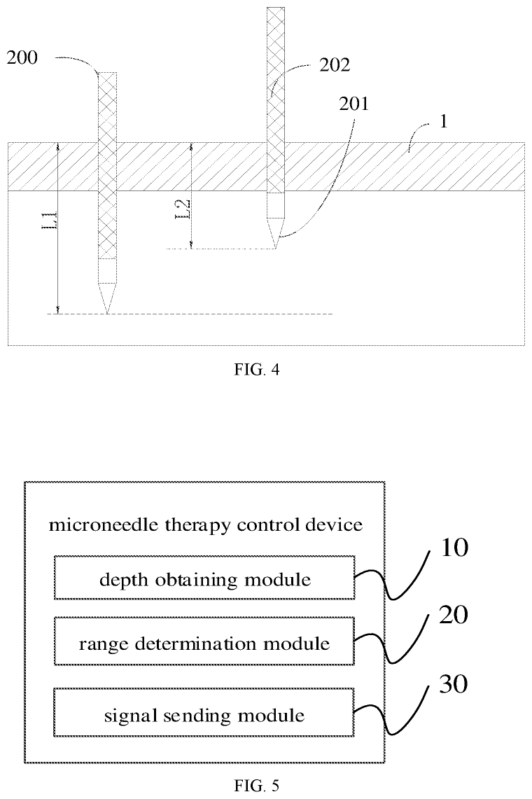

After the microneedle electrode of the radio-frequency therapy instrument penetrates into the target tissue of the human body at a preset depth, the radio-frequency energy is outputted to the target tissue for therapy. As shown in FIG. 4, however, since the needle tip 201 of the microneedle electrode is the energy release area and the rest part of the needle body is coated with an insulating layer 202, when the microneedle electrode penetrates into the human body, only the target tissue adjacent to the needle tip can be treated. In particular, the penetration depth of microneedle electrodes is constant. For target tissues that spread over a long area in the depth direction, the radio-frequency therapy instrument can only treat the target tissue at the penetration depth. For target tissues between the penetration depth and the surface of the human body, the therapy effect is poor.

Therefore, embodiments of the present application provide a radio-frequency microneedle therapy instrument. The radio-frequency microneedle therapy instrument controls a linear driver to drive the microneedle electrode to move along the depth direction of the target tissue, and the microneedle electrode continuously releases radio-frequency energy during the movement process, to effectively treat all parts of the target tissue. In this way, not only the target tissue at the penetration depth can be treated, but also the target tissue at a smaller depth can be treated, thereby improving the effect of radio-frequency therapy.

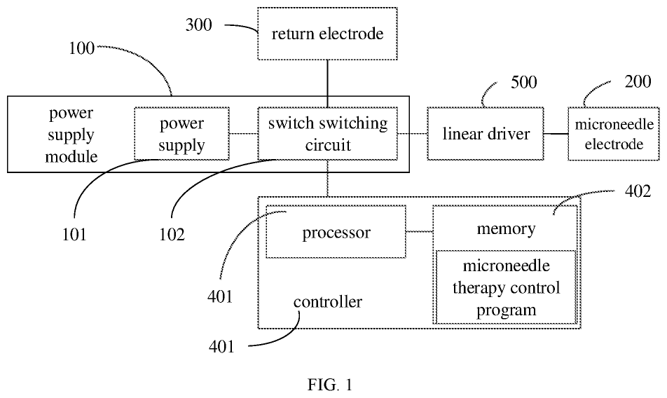

As shown in FIG. 1, FIG. 1 is a schematic structural diagram of a radio-frequency therapy instrument according to the embodiments of the present application.

The radio-frequency microneedle therapy instrument includes a power supply module 100, a microneedle electrode 200, a linear driver 500 and a controller 400.

The power supply module 100 may include a power supply 101 and a switch switching circuit 102. The output frequency of the power supply 101 may range from 0.3 MHz to 100 MHz, and the power supply 101 may be a continuous output power supply, a pulse output power supply, or a continuous and pulse output power supply. The power supply module 100 may include only one power supply 101. That is, a single power supply 101 supplies power to all microneedle electrodes 200. Or the power supply module 100 may include a plurality of the power supplies 101, and the plurality of power supplies 101 are respectively connected to different microneedle electrodes 200. The output power of the power supply 101 is adjustable. The power supply has a preset power mode. The preset power mode includes an increasing power mode, a constant power mode and a decreasing power mode. During actual operation, the power supply can operate in any of these modes.

The switch switching circuit 102 in the power supply module 100 is configured to switch the connection port between the microneedle electrode 200 and the power supply 101, so that the microneedle electrode 200 can be connected to the positive electrode of the power supply 101 to be the positive polarity during the therapy process, or the microneedle electrode 200 can be connected to the negative electrode of the power supply 101 to be the negative polarity.

The plurality of microneedle electrodes 200 are distributed as a microneedle array 200. The microneedle array 200 includes a printed circuit board (PCB) and a plurality of microneedle electrodes 200 disposed at the PCB. The plurality of microneedle electrodes 200 are distributed in an array, such as a 7×7 array. The microneedle electrodes 200 at the microneedle array 200 can be positive electrodes or negative electrodes according to the requirements, and the polarity of each microneedle electrode 200 can be switched to alternately serve as a positive electrode and a negative electrode in different operating periods. Specifically, the microneedle electrode 200 can be connected to different ports of the power supply 101 according to the switch switching circuit 102.

The linear driver 500 can be a linear motor, the microneedle array is fixed at the moving end of the linear motor, and the length direction of the microneedle electrode 200 is consistent with the moving direction of the linear driver 500, so that the microneedle array can move along the length direction of the microneedle electrode 200 back and forth. The linear actuator 500 can further be a push rod or other mechanism, which is not limited here.

The radio-frequency therapy instrument further includes a controller 400. The controller 400 includes at least one processor 401, a memory 402, and a microneedle therapy control program stored in the memory 402 and executable on the processor 401. The microneedle therapy control program is configured to implement the microneedle therapy control method. In some embodiments, the processor 401 and the memory 402 are integrated at the same chip or circuit board. In some other embodiments, the processor 401 and the storage 402, either one or both, can be set at separate chips or circuit boards. That is, the radio-frequency therapy instrument may include the microprocessor such as a single chip, a DSP, and an FPGA. In some embodiments, a dedicated chip may also be provided in the radio-frequency microneedle therapy instrument, which is not limited in this embodiment.

Those skilled in the art can understand that the structure shown in FIG. 1 does not constitute a limitation on the radio-frequency microneedle therapy instrument, and the radio-frequency microneedle therapy instrument may include more or fewer components than shown, or a combination of components, or differently arranged components in the radio-frequency microneedle therapy instrument.

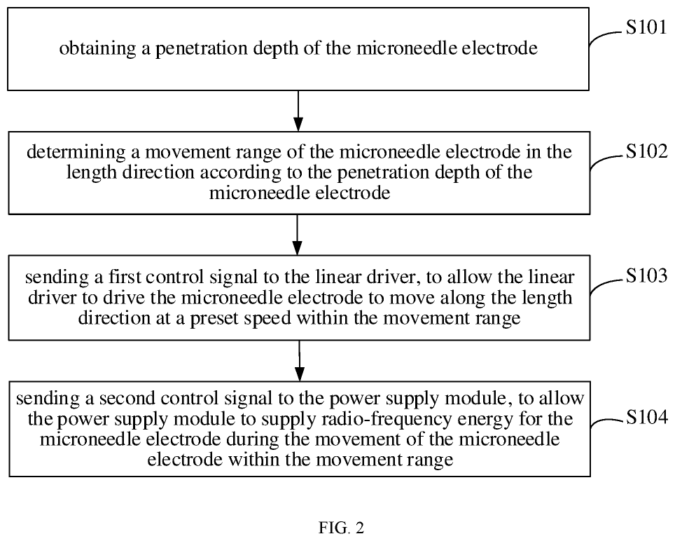

An embodiment of the present application provides a microneedle therapy control method. As shown in FIG. 2. FIG. 2 is a schematic flowchart of a microneedle therapy control method according to a first embodiment of the present application.

In this embodiment, the microneedle therapy control method includes the following operations.

Operation S101, obtaining a penetration depth of the microneedle electrode 200.

In this operation, the microneedle electrode 200 may include one microneedle electrode 200 or several microneedle electrodes 200, or may include all microneedle electrodes 200. The number of microneedle electrodes 200 can be determined according to the therapy part in the human body.

As shown in FIG. 4, the penetration depth of the microneedle electrode 200 is the distance between the needle tip of the microneedle electrode 200 at the part to be treated and the human body surface.

In an embodiment, the penetration depth of the microneedle electrode 200 can be obtained according to a mapping table between the part to be treated and the preset penetration depth of the microneedle electrode 200. For example, the depth mapping table includes penetration depth corresponding the abdomen, the leg, the neck, the armpit and other parts. When using the microneedle electrode 200, the user can obtain the corresponding penetration depth according to the part that the microneedle electrode 200 needs to penetrate into, and then the penetration depth of the microneedle electrode 200 can be set.

Operation S102, determining a movement range of the microneedle electrode 200 in the length direction according to the penetration depth of the microneedle electrode 200.

In this operation, the microneedle electrode 200 has a configured penetration depth L1, that is, the maximum depth at which the needle tip 201 of the microneedle electrode 200 penetrates into the human body. Moreover, after penetrating into the target tissue of the human body, the microneedle electrode 200 will not move left or right. Therefore, the movement range is a range determined by the maximum depth and the minimum depth of the needle tip of the microneedle electrode 200 in the length direction. The microneedle electrode 200 moves along the length direction, and the needle tip moves within the movement range and releases energy.

In an embodiment, operation S102 includes:
determining the minimum energy output depth of the microneedle electrode 200 according to the part to be treated;
determining the movement range of the microneedle electrode 200 in the length direction according to the penetration depth and the minimum energy output depth.

As shown in FIG. 4, the minimum energy output depth L2 is the distance from the needle tip 201 of the microneedle electrode 200 to the human body surface, and is used for preventing the radio-frequency energy delivered by the microneedle electrode 200 from burning the epidermis layer 1. When the distance from the needle tip 201 of the microneedle electrode 200 to the human body surface is less than the minimum energy output depth, the microneedle electrode 200 no longer outputs radio-frequency energy.

Therefore, in the length direction of the microneedle electrode 200, as long as the maximum depth value of the needle tip 201 of the microneedle electrode 200 from the human body surface and the minimum value, namely the penetration depth L1 and the minimum energy output depth L2 are determined, the movement range of the needle electrode 200, namely the energy output range of the needle tip 201 of the needle electrode 200 can be determined.

Operation S103, sending a first control signal to the linear driver 500, to allow the linear driver 500 to drive the microneedle electrode 200 to move along the length direction at a preset speed within the movement range.

Operation S104, sending a second control signal to the power supply module 100, to allow the power supply module 100 to supply radio-frequency energy for the microneedle electrode 200 during the movement of the microneedle electrode 200 within the movement range.

After the movement range of the microneedle electrode 200 is determined, the microneedle electrode 200 can be driven to move within the movement range and output energy. Specifically, the linear driver 500 drives the microneedle electrode 200 to move along the length direction of the microneedle electrode 200 at a preset speed within the movement range, and during this movement process, the power supply module 100 supplies radio-frequency energy for the microneedle electrode 200. The needle tip of the needle electrode 200 outputs radio-frequency energy. That is, the microneedle electrode 200 does not move left or right to avoid damaging tissues in the human body. Besides, the linear driver 500 drives the microneedle electrode 200 to move at a preset speed. For example, the linear driver 500 can be driven to move slowly to prevent the microneedle electrode 200 from moving rapidly within the human tissue and damaging the human tissue. Moreover, it can be ensured that the radio-frequency energy of the microneedle electrode 200 diffuses within the target tissue, thereby achieving corresponding therapy effects.

In this embodiment, the movement of the microneedle electrode 200 within the movement range means that the microneedle electrode 200 may move inwards from the human body surface, or means that the microneedle electrode 200 may penetrate into the human body at the preset penetration depth and then move along the direction from inside the human body to outside the human body, which is not limited here. It should be noted that the penetration depth of the microneedle electrode 200 can be read through the encoder at the linear driver 500, so that the expansion and contraction length of the microneedle electrode 200, that is, the penetration distance from the needle tip of the microneedle electrode 200 to the human body surface, can be monitored in real time.

In an embodiment, when the microneedle electrode 200 moves in a direction outside the human body, operation S104 includes:
determining a preset operating power mode of the power supply module according to a mapping table between a part to be treated and a preset power mode of the power supply module, the preset power mode including an increasing power mode, a constant power mode and a decreasing power mode;
sending the second control signal to the power supply module, to allow the power supply module to operate in the preset power mode and supply radio-frequency energy for the microneedle electrode during the movement of the microneedle electrode within the movement range.

Specifically, the power supply 101 can operate in a preset power mode according to whether the therapy part is the armpit, the face or the back. For example, in regard to facial tightening, to avoid damaging facial skin and causing disfigurement due to too high radio-frequency energy output power, the power supply 101 in the power module 100 can operate in a gradually decreasing power mode, that is, the output power decreases progressively.

In this embodiment, by controlling the linear driver 500 to drive the microneedle electrode 200 to move along the depth direction of the target tissue, and the microneedle electrode 200 continuously releases radio-frequency energy during the movement, various parts of the target tissue can be effectively treated. In this way, not only target tissues at a larger depth can be treated, but also target tissues at a smaller depth can be treated, resulting in a wider therapy area, thereby improving the effect of radio-frequency therapy.

Based on the first embodiment of the microneedle therapy control method of the present application, a second embodiment of the microneedle therapy control method of the present application is proposed. In this embodiment, operation S104 includes:
sending the second control signal to the power supply module 100, to allow the power supply module 100 to supply one of a first electric polarity and a second electric polarity for at least one of the at least two microneedle electrodes 200 during the movement of the microneedle electrode 200 within the movement range, and to allow the power supply module 100 to supply another one of the first electric polarity and the second electric polarity for remaining microneedle electrodes 200 of the at least two microneedle electrodes 200.

Specifically, during the therapy process, a plurality of microneedle electrodes 200 penetrate into the part of the human body to be treated, and then during the movement of the microneedle electrodes 200, at least one of the plurality of microneedle electrodes 200 is a positive electrode, and the rest are negative electrodes, thereby forming a thermal dispersion area between the positive microneedle and the negative microneedle. In this case, the first electrode polarity is the positive electrode and the second electrode polarity is the negative electrode. For example, only one microneedle electrode 200 of the plurality of microneedle electrodes 200 is the positive electrode.

Or at least one of the plurality of microneedle electrodes 200 is a negative electrode, and the rest are positive electrodes. A thermal diffusion area is formed between the positive electrodes and the negative electrodes. For example, only one microneedle electrode 200 of the plurality of microneedle electrodes 200 is a negative electrode. In this embodiment, the positive electrode and the negative electrode in the plurality of microneedle electrodes 200 are not arranged in one-to-one correspondence.

In this embodiment, compared to the microneedle array in which the positive electrode and the negative electrode are arranged in one-to-one correspondence, at least one microneedle electrode 200 in the microneedle array has the polarity opposite to the polarity of the rest microneedle electrodes 200, so that during therapy process, energy flows from a smaller number of microneedle electrodes 200 with the first electric polarity to a larger number of microneedle electrodes 200 with the second electric polarity. Or, during the process that energy flows from a smaller number of microneedle electrodes 200 with the second electric polarity to a larger number of microneedle electrodes 200 with the first electric polarity, the thermal dispersion area of the microneedle array has a larger lateral range, so that a better therapy effect can be achieved.

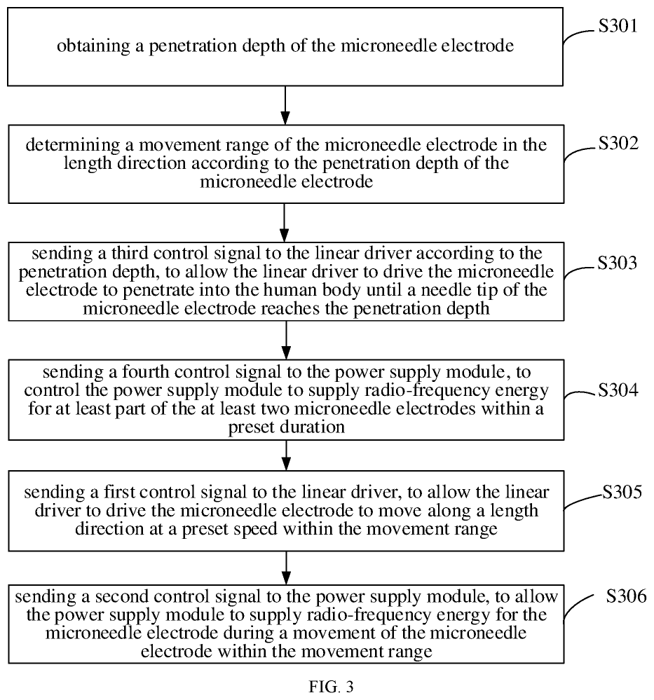

Based on the above-mentioned first embodiment and second embodiment of the microneedle therapy control method of the present application, a third embodiment of the microneedle therapy control method of the present application is proposed. As shown in FIG. 3, FIG. 3 is a schematic flowchart of the microneedle therapy control method according to a second embodiment of the present application.

In this embodiment, the following operations are included.

Operation S301, obtaining a penetration depth of the microneedle electrode 200.

Operation S302, determining a movement range of the microneedle electrode 200 in the length direction according to the penetration depth of the microneedle electrode 200.

Operation S303, sending a third control signal to the linear driver 500 according to the penetration depth, to allow the linear driver 500 to drive the microneedle electrode 200 to penetrate into the human body until a needle tip of the microneedle electrode 200 reaches the penetration depth.

Specifically, the linear driver 500 drives the microneedle electrodes 200 in the microneedle array penetrate into the human body, and the depth at which the microneedle electrodes 200 penetrates into the human body is a preset penetration depth. During this process, the microneedle electrode 200 does not release energy.

Operation S304, sending a fourth control signal to the power supply module 100, to control the power supply module 100 to supply radio-frequency energy for at least part of the at least two microneedle electrodes 200 within a preset duration.

Specifically, after the microneedle electrode 200 penetrates into the human body, radio-frequency energy can be output within a preset duration according to the conventional radio-frequency therapy method, to treat the target tissue near the needle tip. In this case, the power supply module 100 can supply radio-frequency energy for all microneedle electrodes 200 and can also supply radio-frequency energy for partial microneedle electrodes 200. It can be understood that the therapy will be carried out after the microneedle electrode 200 penetrates into the corresponding penetration depth, which is an existing technology and will not be described here.

In an embodiment, the radio-frequency microneedle therapy instrument also includes a return electrode disposed on the body surface, and the return electrode is electrically connected to the power supply module 100.

In operation S304, the microneedle array may be in the unipolar mode. The fourth control signal is sent to the power supply module 100, to keep the power supply module 100 in the unipolar mode within the preset duration, supply one of the first electric polarity and the second electric polarity for the at least two microneedle electrodes 200, and supply another one of the first electric polarity and the second electric polarity for the return electrode.

The return electrode is attached to the surface of the human body. In this case, the electric polarity of all microneedle electrodes 200 in the microneedle array is the same and is opposite to the electric polarity of the return electrode. For example, when all the microneedle electrodes 200 in the microneedle array are of the first electric polarity, the return electrode is of the second electric polarity, thereby forming a loop between the deep part of the human body and the surface of the human body, resulting a larger depth coverage of the heat dispersion area to improve the radio-frequency therapy effect. Or all microneedle electrodes 200 in the microneedle array may be of the second electric polarity, and the return electrode is of the first electric polarity.

The first electric polarity may be positive and the second electric polarity may correspondingly be negative.

In operation S304, the microneedle array may also be in the bipolar mode. The fourth control signal is further configured to trigger the power supply module 100 to be in a bipolar mode during a conversion duration, to supply one of the first electric polarity and the second electric polarity for at least one of a plurality of microneedle electrodes 200 and supply another one of the first electric polarity and the second electric polarity for remaining microneedle electrodes 200 of the plurality of the microneedle electrodes 200. The conversion duration is a part of the preset duration, and the unipolar mode and the bipolar mode are switched at least once within the preset duration.

In the unipolar mode, the thermal dispersion range of the microneedle electrode 200 is larger in the depth direction, but the loop is formed between inside and outside the body, that is, the thermal dispersion range is narrow in the lateral direction. To improve the therapy effect of the part near the penetration depth, that is, to make the heat dispersion range near the penetration depth wider in the lateral direction. Further, a bipolar mode is provided in this embodiment. In the bipolar mode, the return electrode is not in the loop, and some of the microneedle electrodes 200 in the microneedle array are positive electrodes. In an embodiment, one microneedle electrode 200 is the positive electrode, and the remaining electrodes are negative electrodes. Or some of the microneedle electrode 200 are the negative electrode. In an embodiment, one microneedle electrode 200 is the negative electrode, and the remaining microneedle electrodes 200 are the positive electrodes. The number of positive electrodes is inconsistent with the number of negative electrodes, and a thermal dispersion area is formed between the positive microneedle electrode 200 and the negative microneedle electrode 200, resulting a wider dispersion range of the radio-frequency energy in the lateral direction.

In this embodiment, the unipolar mode and the bipolar mode are switched at least once within the preset duration, thereby improving the radio-frequency therapy effect when the microneedle array penetrates into the human body at a constant depth.

Operation S305, sending a first control signal to the linear driver 500, to allow the linear driver 500 to drive the microneedle electrode 200 to move in an outward direction from a human body at a preset speed within the movement range.

Operation S306, sending a second control signal to the power supply module 100, to allow the power supply module 100 to supply radio-frequency energy for the microneedle electrode 200 during a movement of the microneedle electrode 200 within the movement range.

After performing radio-frequency therapy at a conventional depth for a period of time, the microneedle electrode 200 needs to be pulled out of the human body. In this embodiment, the linear driver 500 drives the microneedle electrode 200 to be pulled out from the human body slowly, to output radio-frequency energy during the pull-out process, thereby also performing radio-frequency therapy on the human tissue during the pull-out process.

In this embodiment, firstly, the microneedle electrode 200 penetrates into the human body at a preset penetration depth, and radio-frequency therapy is performed at the penetration depth. The therapy duration is the preset duration. That is, conventional radio-frequency therapy with a constant depth is first performed. Then, after the conventional radio-frequency therapy with the constant depth is completed, the microneedle electrode 200 is pulled out slowly, and during this process, the microneedle electrode 200 continues to output energy. Thus, radio-frequency therapy for the target tissue within the movement range is completed. Compared with the existing radio-frequency therapy that only performs radio-frequency therapy near the preset penetration depth, the therapy range in this embodiment is wider in the depth direction, that is, the therapy effect is better.

As shown in FIG. 5, FIG. 5 is a structural block diagram of the microneedle therapy control device according to the first embodiment of the present application.

As shown in FIG. 5, the microneedle therapy control device includes:
a depth obtaining module 10, configured to obtain a penetration depth of a microneedle electrode 200;
a range determination module 20, configured to determine a movement range of the microneedle electrode 200 in a length direction according to the penetration depth of the microneedle electrode 200; and
a signal sending module 30, configured to send a first control signal to a linear driver 500 to allow the linear driver 500 to drive the microneedle electrode 200 to move along a length direction at a preset speed within the movement range, and send a second control signal to a power supply module 100 to allow the power supply module 100 to supply radio-frequency energy for the microneedle electrode 200 during the movement of the microneedle electrode 200 within the movement range.

The above are only some embodiments of the present application, and do not limit the scope of the present application thereto. Under the concept of this application, any equivalent structural transformation made according to the description and drawings of the present application, or direct/indirect application in other related technical fields shall fall within the claimed scope of the present application.

What is claimed is:

1. A microneedle therapy control method, applied to a radio-frequency microneedle therapy instrument, wherein the radio-frequency microneedle therapy instrument comprises:
at least two microneedle electrodes movable along a length direction of the microneedle electrodes;
a power supply module configured to supply radio-frequency energy for the microneedle electrode; and
a linear driver configured to drive the microneedle electrode to move,
wherein the method comprises:
obtaining a penetration depth of the microneedle electrode;
determining a movement range of the microneedle electrode in the length direction according to the penetration depth of the microneedle electrode;
sending a first control signal to the linear driver, to allow the linear driver to drive the microneedle electrode to move along the length direction at a preset speed within the movement range; and
sending a second control signal to the power supply module, to allow the power supply module to supply radio-frequency energy for the microneedle electrode during a movement of the microneedle electrode within the movement range.

2. The microneedle therapy control method of claim 1, wherein the sending the second control signal to the power supply module, to allow the power supply module to supply radio-frequency energy for the microneedle electrode during the movement of the microneedle electrode within the movement range comprises:
sending the second control signal to the power supply module, to allow the power supply module to supply one of a first electric polarity and a second electric polarity for at least one of the at least two microneedle electrodes during the movement of the microneedle electrode within the movement range, and allow the power supply module to supply another one of the first electric polarity and the second electric polarity for remaining microneedle electrodes of the at least two microneedle electrodes.

3. The microneedle therapy control method of claim 1, wherein the sending the first control signal to the linear driver, to allow the linear driver to drive the microneedle electrode to move along the length direction at the preset speed within the movement range comprises:
sending the first control signal to the linear driver, to allow the linear driver to drive the microneedle electrode to move in an outward direction from a human body at a preset speed within the movement range.

4. The microneedle therapy control method of claim 3, wherein the sending the second control signal to the power supply module, to allow the power supply module to supply radio-frequency energy for the microneedle electrode during the movement of the microneedle electrode within the movement range comprises:
determining a preset operating power mode of the power supply module according to a mapping table between a part to be treated and a preset power mode of the power supply module, wherein the preset power mode comprises an increasing power mode, a constant power mode and a decreasing power mode; and
sending the second control signal to the power supply module, to allow the power supply module to operate in the preset power mode and supply radio-frequency energy for the microneedle electrode during the movement of the microneedle electrode within the movement range.

5. The microneedle therapy control method of claim 3, wherein before the sending the first control signal to the linear driver, to allow the linear driver to drive the microneedle electrode to move along the length direction at the preset speed within the movement range, the method further comprises:
sending a third control signal to the linear driver according to the penetration depth, to allow the linear driver to drive the microneedle electrode to penetrate into the human body until a needle tip of the microneedle electrode reaches the penetration depth; and
sending a fourth control signal to the power supply module, to allow the power supply module to supply radio-frequency energy for at least part of the at least two microneedle electrodes within a preset duration.

6. The microneedle therapy control method of claim 5, wherein:
the radio-frequency microneedle therapy instrument further comprises a return electrode arranged at a body surface, the return electrode is electrically connected to the power supply module; and
the sending the fourth control signal to the power supply module, to control the power supply module to supply radio-frequency energy for at least part of the at least two microneedle electrodes within the preset duration comprises:
sending the fourth control signal to the power supply module, to keep the power supply module in a unipolar mode within the preset duration to supply one of a first electric polarity and a second electric polarity for the at least two microneedle electrodes and supply another one of the first electric polarity and the second electric polarity for the return electrode.

7. The microneedle therapy control method of claim 6, wherein the fourth control signal is further configured to trigger the power supply module to be in a bipolar mode during a conversion duration, to supply one of the first electric polarity and the second electric polarity for at least one of a plurality of microneedle electrodes and supply another one of the first electric polarity and the second electric polarity for remaining microneedle electrodes of the plurality of the microneedle electrodes, the conversion duration is a part of the preset duration, and the unipolar mode and the bipolar mode are switched at least once within the preset duration.

8. The microneedle therapy control method of claim 1, wherein the determining the movement range of the microneedle electrode in the length direction according to the penetration depth of the microneedle electrode comprises:
determining a minimum energy output depth of the microneedle electrode according to the part to be treated; and
determining the movement range of the microneedle electrode in the length direction according to the penetration depth and the minimum energy output depth.

9. The microneedle therapy control method of claim 8, wherein the obtaining the penetration depth of the microneedle electrode comprises:
obtaining the penetration depth of the microneedle electrode according to a mapping table between the part to be treated and a preset penetration depth of the microneedle electrode.

10. A microneedle therapy control device, comprising:
a depth obtaining module, configured to obtain a penetration depth of a microneedle electrode;
a range determination module, configured to determine a movement range of the microneedle electrode in a length direction according to the penetration depth of the microneedle electrode; and
a signal sending module, configured to send a first control signal to a linear driver to allow the linear driver to drive the microneedle electrode to move along a length direction at a preset speed within the movement range, and send a second control signal to a power supply module to allow the power supply module to supply radio-frequency energy for the microneedle electrode during a movement of the microneedle electrode within the movement range.

11. A radio-frequency microneedle therapy instrument, comprising:
at least two microneedle electrodes movable along a length direction of the microneedle electrodes;
a power supply module configured to supply radio-frequency energy for the microneedle electrode;
a linear driver configured to drive the microneedle electrode to move; and
at least one processor, a memory, and a microneedle therapy control program stored in the memory and executable on the processor, wherein when the microneedle therapy control program is executed by the processor, the microneedle therapy control method of claim 1 is implemented.
